(19) United States
(12) Patent Application Publication
(10) Pub. No.: US 2007/0282241 A1
(43) Pub. Date: Dec. 6, 2007

(54) Applicator system

(76) Inventor: Meryl J. Squires, Barrington Hills, IL (US)

Correspondence Address: Welsh & Katz, Ltd., 22nd Floor, 120 South Riverside Plaza, Chicago, IL, 60606-3945, US

(21) Appl. No.: 11/443,582

(22) Filed: May 31, 2006

(51) Int. Cl. A61M 35/00

(52) U.S. Cl. 604/2; 604/295

(57) ABSTRACT

An applicator system and assembly is provided with a user-friendly medical applicator device and closure cap for easy and effective application of medicine or other material. The applicator device comprises an ergonomically designed front applicator portion, a special intermediate applicator portion, and a multi-use handle.

### BACKGROUND OF THE INVENTION

This invention pertains to an applicator and, more particularly, to a medical device, for applying medicine or other material.

In the past, many different medical devices and instruments has been developed to facilitate the application or insertion of medicine into persons or animals inflicted with pain or disease. Many of the prior medical devices and instruments are cumbersome, awkward, uncomfortable, difficult to use, expensive, undependable, inefficient, and/or ineffective. Furthermore, some of the prior medical devices and instruments require o rings or gaskets for tight seals which are not cost effective and/or leak. These prior medical devices and instruments have met with varying degrees of success.

It is, therefore, desirable to provide an improved applicator system and assembly which overcomes most, if not all, of the preceding problems.

### BRIEF SUMMARY OF THE INVENTION

An improved applicator system and assembly is provided with a novel medical applicator device and special cap which are easy to use, convenient, effective, reliable and safe. The user friendly comfortable applicator device comprises a front applicator portion, an intermediate applicator portion, and a handle. The cap is constructed and arranged to receive the front and intermediate applicator portions of the applicator device. The efficient applicator device and the cap can comprise different materials.

In the illustrative embodiment, the cap snugly receives the front and intermediate applicator portions of the applicator device, and comprises an elongated tube providing a container to contain and hold material, such as medicine, a pharmaceutical product(s), a compound(s), or a composition.

The front applicator portion can comprise a finger with a base and a convex rounded tip for engaging the cap. The finger can extend forwardly of the base and the intermediate applicator portion to apply the material in the container in oral mucosa, nasal mucosa, vagina tissue, labial tissue, rectal tissue, anal tissue, peri-anal tissue, lips, cutaneous tissue, ocular tissue, conjunctiva, eyelids, mouth, lymph nodes, or other crevices or openings in a person, animal, bird, or fish.

In the illustrated embodiment, the intermediate applicator portion is positioned between and is connected to the front applicator portion and the handle. The intermediate portion can have a central body that provides a core and at least one rib and a chamber, and preferably has at least two ribs, that extend radially outwardly from the body past a maximum radial span of the finger of the front applicator portion. The ribs can comprise substantially parallel elongated longitudinal fins which span an axial length of the body of the intermediate applicator portion.

A more detailed explanation of the invention is provided in the following description and appended claims taken in conjunction with the accompanying drawings.

### BRIEF DESCRIPTION OF THE DRAWINGS

FIG. 1 is a perspective view of an applicator system and assembly in accordance with principles of the present invention;

FIG. 2 is a front view of the applicator system and assembly;

FIG. 3 is a right side view of the applicator system and assembly;

FIG. 4 is a back view of applicator system and assembly;

FIG. 5 is a left side view applicator system and assembly;

FIG. 6 is top plan view of the applicator system and assembly; and

FIG. 7 is a bottom view of the applicator system and assembly.

### DETAILED DESCRIPTION OF THE INVENTION

A detailed description of the preferred embodiment and best mode for practicing the invention are discussed herein.

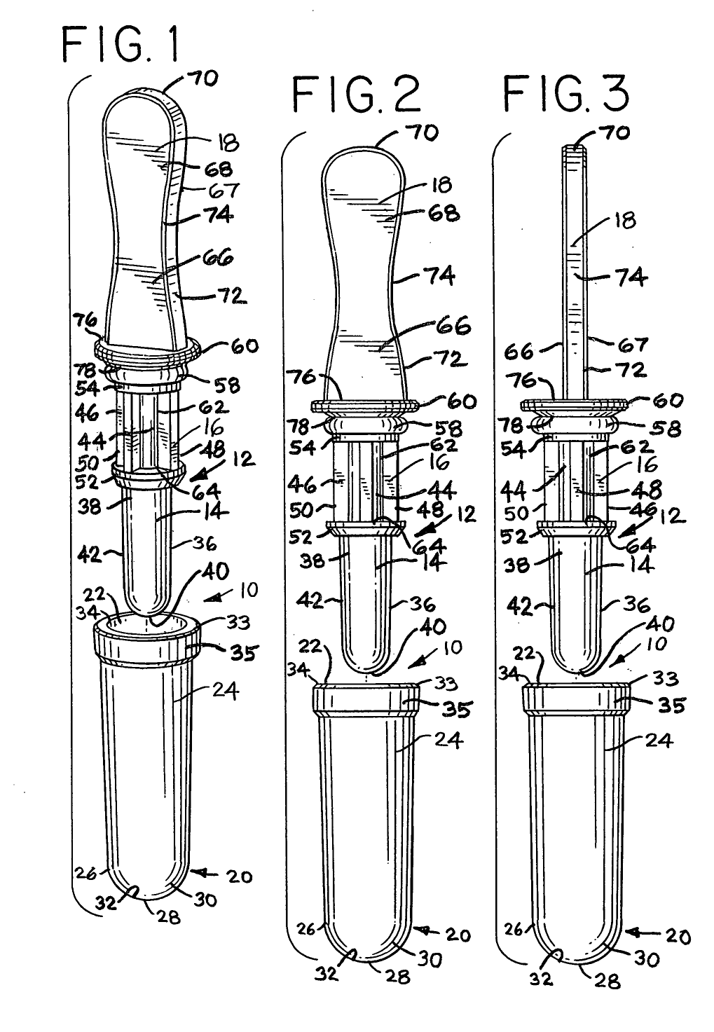

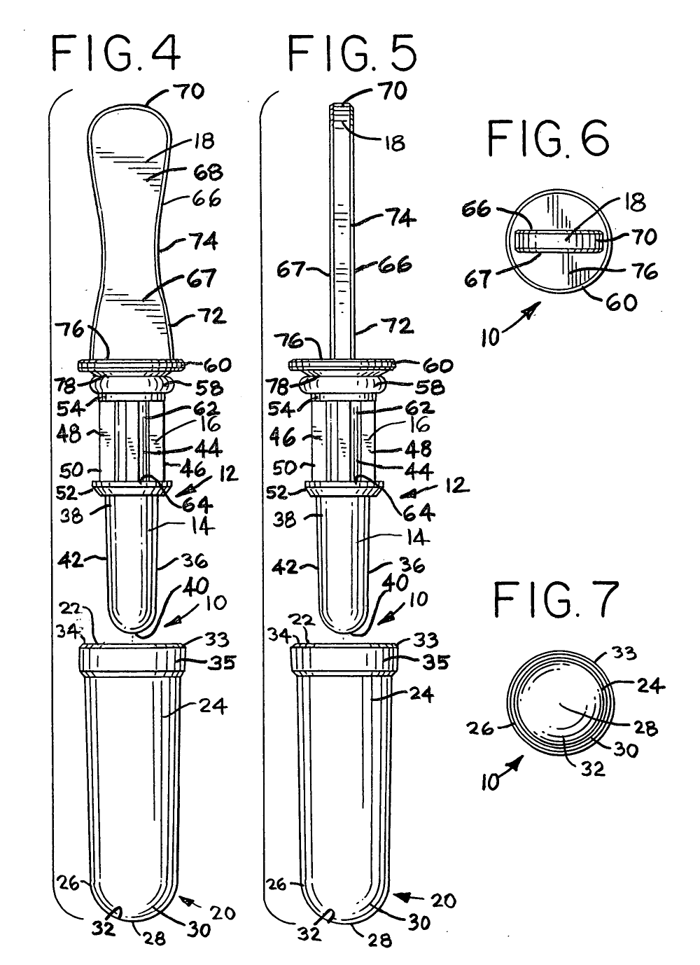

An applicator system and/or assembly 10 (FIGS. 1-7) provides a unit dose (doseage) applicator that is user friendly, comfortable, convenient, and effective. The applicator provides an medical applicator device 12 comprising an ergonomic front applicator portion 14, an intermediate applicator portion 16, and a multi-use handle 18. The applicator system and assembly includes an applicator cap 20 for comfortably, snugly, and/or snap-fittingly receiving, covering and closing the front and intermediate applicator portions of the applicator device when the applicator is in the storage position. The cap 20 can be removed when the applicator is in the open applicating position for insertion and/or engaging the area to be treated and/or applying a material 22, such as medicine, a pharmaceutical product(s), a compound(s), and a composition.

The cap comprises an elongated tube 24 providing a container 26 for containing and holding the material, such as medicine, a pharmaceutical product(s), a compound(s), and a composition. The cap has a rounded closed end portion 28 with a convex external surface 30 and a concave internal surface 32 that closes one end of the tube. The cap comprises an open end portion 33 that defines and provides an open mouth 34 which is positioned longitudinally opposite the closed end portion. The cap can have a circumferential manually graspable flange 35 that extends radially outwardly of the tube about the open end portion in proximity to and adjacent the mouth.

In the illustrative embodiment, the front applicator portion can be cantilevered from and connected to the intermediate applicator portion. The front applicator can be coaxially aligned with the cap when the cap receives covers and closes the front applicator portion. The front applicator portion can comprises a finger 36 with a base 38 and a convex rounded tip 40 for detachably and removably engaging the cap. The finger can provide a male member and can extend forwardly of the base and the intermediate applicator portion. The finger can have sloping sides 42 providing a frustoconical side section that converges towards the rounded tip. The finger can be useful for inserting and applying the material from the container in oral mucosa, nasal mucosa, vagina tissue, labial tissue, rectal tissue, anal tissue, peri-anal tissue, lips, cutaneous tissue, ocular tissue, conjunctiva, eyelids, mouth, lymph nodes, or other crevices or openings in a person, animal, bird, or fish.

The intermediate applicator portion can extend substantially between the front applicator portion and the handle and can be connected to the front applicator portion and the handle. The intermediate portion can have a central body 44 comprising a core 46 and can have at least one pair of diametrically opposite lateral ribs 46 and 48, preferably two pair of aliquot uniformly spaced diametrically opposite lateral ribs, which extend radially outwardly from the body past a maximum radial span or maximum diameter of the finger of the front applicator portion. The ribs can comprise pairs of substantially parallel elongated longitudinal fins 50 which span an axial length of the body of the intermediate applicator portion. A flared skirt 52 can extend radially outwardly from the base of the front applicator portion past the maximum diameter of the diametrically opposite ribs and diverge in a direction away from the tip of the front applicator portion. A circular rib-engaging disc 54 that provides an intermediate circular flange can abut against the ribs. The circular rib-engaging disc can be positioned between the handle and the rib. An intermediate base flange 58 that provides a grommet can be disposed and positioned between a handle-engaging disc 60 and the rib-engaging disc. The grommet can have a diameter greater than the rib-engaging disc. In the illustrative embodiment, the intermediate applicator portion cooperates with the ribs, skirt, and disk to provide at least two, and preferably four, longitudinal chambers 62 that define pockets 64, which extend substantially the length of the body of the intermediate applicator portion to receive material from the container. The pockets can comprise air pockets or cavities to help seal the applicator device of the cap when the applicator assembly is in the closed position. The chambers also comprise material-receiving chambers to receive the material in the container.

In the illustrative embodiment, the handle is coaxially aligned with the front and intermediate applicator portions of the applicator device. The handle can be gripped and can be used to mix or apply material, and can be used for other purposes. The handle can have a length greater than finger of the front intermediate portion. The length of the handle can also be greater (longer) than the axial length of the intermediate applicator portion of the applicator device. In the illustrative embodiment, the length of the handle is equal to about the total combined length of the front applicator portion and the intermediate applicator portion. The handle can be flexible, generally flat and/or coplanar with a thickness less than the maximum diameter of the intermediate applicator portion. The thickness of the handles can also be less than a maximum lateral span or maximum diameter of the finger of the front applicator portion. The flat surfaces 66-67 of the handle can have an identification section 68 comprising a raised portion, an embossed portion, or a flat portion with print containing a name, trademark, logo, insignia, design, bar code, identification, and/or instructions. The handle can comprise a rounded end section 70, a flared section 72 that diverges towards the intermediate applicator section, and a manually graspable central section 74 that is disposed and positioned between and connected to the rounded end section and the flared section. The central section of the handle can have a maximum lateral span less than the maximum lateral span of the rounded end section of the handle. The maximum lateral span of the central section of the handle can also be less than the maximum lateral of the flared section of the handle. The applicator device can also have a circular handle-engaging disc 60 with a diameter greater than a maximum width or lateral span of the handle. The circular handle-engaging disc can have a handle-engaging surface 76 to abuttingly engage i.e. that abuts against, the end portion of the flared section of the handle and can have an intermediate applicator portion-facing surface 78 that provides a seat for engaging and supporting the cap when the cap closes, covers and receives the front and intermediate portions of the applicator device in the closed storage position.

Preferably, the applicator device and the cap comprise different materials to provide a better cost-effective seal when the application is in the closed storage position. The applicator device can be made of one or more of the following: a resilient material, an elastomeric material, a flexible material, medical grade plastic or other plastic, or rubber. Such material can include, but are not limited to, polyvinyl chloride (PVC), neoprene, santoprene, urethane, etc. In some circumstances, it may be desirable to use wood or metal for part or all of the applicator device. The medical grade plastic can comprise an FDA (U.S. Food & Drug Administration approved) grade thermoplastic elastomer (TPE), such as Krayton brand plastic. Other types of plastic can be used. The cap can comprise polypropylene, medical grade plastic or other plastics, or metal, such as stainless steel.

The material contained in the cap can comprise a composition, such as a topical analgesic with therapeutic benefits for use in humans, equine, bovine, canine, feline, porcine, and other animals and birds and/or for treatment and healing of ligaments, tendons, muscles, bones, hematomas, nerves, and sports injuries and/or for epidermal and dermal treatment of arthritis, neuralgia and pain.

The material contained in the cap can comprise a veterinary medicine for treating viral or bacterial infections or infectious diseases in dogs, cats, birds, horses, cows, sheep, swine (pigs or hogs), or other farm animals, rodents, or animals in zoos.

The material contained in the cap can comprise a composition in the form of a solution, ointment, cream, lubricant, coating, mixture of chemicals, or another type of composition.

In one form, the material contained in the cap comprises a pharmaceutical product comprising a muscle relaxant.

In another form, the material contained in the cap comprises a pharmaceutical product including a pharmaceutical preparation with antiviral, and/or antimicrobial properties for herpes related microbial infections, such as for: cold sores, herpes simplex virus, varicella zoster virus (herpes zoster) (shingles), and cytomegalovirus, and/or for use in the treatment of a viral disease selected from the group consisting of herpetic keratitis, conjunctivitis, viral influenza (flu), herpes simplex virus 1 (HSV 1), herpes simplex virus 2 (HSV 2), esptein barr, papilloma virus, viral parainfluenza, adenovirus, viral encephalitis, viral menigitus, arbovirus, arenavirus, picomavirus, coronavirus, syntialvirus, viral infections, common colds caused by viruses, human immunedeficiency virus (HIV), HIV retinopathy, geniculate zoster, chicken pox, roseola infantum, organ transplant rejection, pneumonia, infectious mononucleosis, uveitis, retinitis, human cervical carcinoma, vaginal carcinoma, vulvovaginitis, human herpes IV, and Kaposi's sarcoma.

The material contained in the cap can further comprise a medicine for inhibiting HIV, herpes, viral infections or other infectious diseases in human beings. Appropriate medicine can be used in varying dilutions on: oral and nasal mucosa; vaginal tissue; labial tissue; anal and peri-anal tissue; penile tissue; cutaneous tissue; open subcutaneous tissue; and in higher dilutions on ocular infections. The medical solution (medicine) can be applied systemically, vaginally, rectally, or in another manner. By varying the concentrations, the medicine may possibly be administered parenterally.

The use of the applicator with the appropriate medicine can provide a medical treatment that can resolve the physical symptoms of an infectious outbreak of HIV, herpes simplex virus 1 or 2 (HSV 1 or HSV 2) or other infectious microbial diseases. The preceding can be accomplished by systemically applying or injecting the above described preferred antimicrobial compound (medicine) with the applicator device into the rectal canal (rectum, rectal tissue, anus or anal tissue) or the vagina (vaginal tissue) of a patient infected with HIV or other infectious microbial disease, preferably numerous times during the day, at regular intervals, for consecutive days, such as 8-12 times per day, preferably 10 times a day at intervals of every two hours, for a period of 10-18 consecutive days, preferably 14 consecutive days (two weeks) for best results. The dosage, concentration, and amount of the antimicrobial compound (medicine) can be varied depending on the severity and extent of the disease as well as the age, sex, weight, race and health of the patient. Desirably, the infected area is rinsed (washed) and dried to remove any soap or residue on the infected area before the antimicrobial compound (medicine) is applied. For treatment of herpes simplex virus 1 or 2, the antimicrobial compound can be applied on the infected area, such as for 19-24 hours. Preferably, vesicular eruption of herpes virus can be resolved in 19-24 hours and herpes lesions can be consequently healed.

Among the many advantages of the preceding applicator system and assembly are:
1. Superb treatment.
2. Superior results in minimizing and relieving pain.
3. Outstanding performance.
4. Convenient.
5. Comfortable.
6. Reliable.
7. Flexible.
8. Resilient.
9. Non-abrasive.
10. Attractive.
14. Easy to use.
15. Portable.
16. Light weight.
17. Soft.
18. Safe.
19. Economical.
20. Efficient.
21. Effective.

Although embodiments of the invention have been shown and described, it is to be understood that various modifications and substitutions, different materials, as well as rearrangements of parts, components, and equipment, methods, and uses, can be made by those skilled in the art without departing from the novel spirit and scope of this invention.

What is claimed is:

1: An applicator assembly, comprising:
an applicator device comprising a front applicator portion, an intermediate applicator portion, and a handle; and
a cap for receiving the front and intermediate applicator portions of the applicator device.

2: An applicator assembly in accordance with claim 1 wherein said cap comprises an elongated tube providing a container for containing material selected from the group consisting of medicine, a pharmaceutical product, a compound, and a composition.

3: An applicator assembly in accordance with claim 2 wherein said cap comprises:
a rounded closed end portion with a convex external surface and a concave internal surface closing one end of the tube;
an open end portion defining an open mouth positioned longitudinally opposite the closed end portion; and
a circumferential flange extending radially outwardly of the tube about the open end portion.

4: An applicator assembly in accordance with claim 1 wherein said front applicator portion provides a finger with a base and a convex rounded tip for engaging the cap, and said finger extends forwardly of said base and said intermediate applicator portion.

5: An applicator assembly in accordance with claim 4 wherein said front applicator portion is cantilevered form the intermediate applicator portion and is coaxially aligned with the cap when the cap receive the front applicator portion, and said front applicator portion has sloping sides converging towards said rounded tip.

6: An applicator assembly in accordance with claim 1 wherein:
said intermediate applicator portion is positioned between and is connected to said front applicator portion and said handle; and
said intermediate portion has a central body providing a core and at least one lateral rib extending outwardly from said body.

7: An applicator assembly in accordance with claim 6 wherein:
said rib comprises an elongated longitudinal fin spanning an axial length of said body of said intermediate applicator portion; and
said rib extends radially outwardly past a maximum radial span of said front applicator portion.

8: An applicator assembly in accordance with claim 1 including:
a flared skirt extending radially outwardly from the front applicator portion past a maximum radial span of the rib and diverging in a direction away from the front applicator portion;
a circular disc abutting against the rib and positioned between the handle and the rib; and
said intermediate applicator portion cooperating with said rib, skirt, and disk for providing at least one chamber.

9: An applicator assembly in accordance with claim 1 wherein:
said handle is coaxially aligned with said front and intermediate applicator portions;
said handle has a length greater than said front intermediate portion; and
the length of said handle is greater than an axial length of said intermediate applicator portion.

10: An applicator assembly in accordance with claim 1 wherein:
said handle is generally flat with a thickness less than a maximum lateral span of said intermediate applicator portion; and
the thickness of said handless is less than a maximum lateral span of said front applicator portion.

11: An applicator assembly in accordance with claim 1 including a circular handle-engaging disc having a diameter greater than a maximum width of said handle and providing a seat for said cap.

12: An applicator assembly in accordance with claim 1 wherein said applicator device and said cap comprise different materials.

13: An applicator assembly, comprising:
an applicator device comprising a front applicator portion, an intermediate applicator portion, and a handle; and
a cap for snugly receiving the front and intermediate applicator portions of the applicator device, said cap comprising an elongated tube providing a container for containing material selected from the group consisting of medicine, a pharmaceutical product, a compound, and a composition;
said front applicator portion providing a finger with a base and a convex rounded tip for engaging the cap, and said finger extending forwardly of said base and said intermediate applicator portion for applying the material in the container in oral mucosa, nasal mucosa, vagina tissue, labial tissue, rectal tissue, anal tissue, peri-anal tissue, lips, cutaneous tissue, ocular tissue, conjunctiva, eyelids, mouth, lymph nodes, or other crevices or openings in a person, animal, bird, or fish;
said intermediate applicator portion is positioned between and is connected to said front applicator portion and said handle, said intermediate portion having central body providing a core and at least two ribs extending radially outwardly from said body past a maximum radial span of said finger of said front applicator portion; and said ribs comprising substantially parallel elongated longitudinal fins spanning an axial length of said body of said intermediate applicator portion.

14: An applicator assembly in accordance with claim 13 where said ribs comprise at least one pair of diametrically opposite lateral ribs and at least two chambers defining pockets for facilitating sealing of said cap and said applicator device.

15: An applicator assembly in accordance with claim 13 wherein said applicator device and said cap comprise different materials.

16: An applicator assembly, comprising:
an applicator device comprising a front applicator portion, an intermediate applicator portion, and a handle;
a cap for receiving the front and intermediate applicator portions of the applicator device, said cap comprising an elongated tube providing a container for containing material selected from the group consisting of medicine, a pharmaceutical product, a compound, and a composition, a rounded closed end portion with a convex external surface and a concave internal surface closing one end of the tube, said cap comprising an open end portion defining an open mouth positioned longitudinally opposite the closed end portion, and a circumferential manually graspable flange extending radially outwardly of the tube about the open end portion in proximity to said mouth;
said front applicator portion comprising a finger with a base and a convex rounded tip for detachably engaging the cap, and said finger extending forwardly of said base and said intermediate applicator portion for inserting and applying the material in the container in oral mucosa, nasal mucosa, vagina tissue, labial tissue, rectal tissue, anal tissue, peri-anal tissue, lips, cutaneous tissue, ocular tissue, conjunctiva, eyelids, mouth, lymph nodes, or other crevices or openings in a person, animal, bird, or fish, said front applicator portion being cantilevered from the intermediate applicator portion and being coaxially aligned with the cap when the cap receives the front applicator portion, and said front applicator portion has sloping sides converging towards said rounded tip;
said intermediate applicator portion extending substantially between and connected to said front applicator portion and said handle, said intermediate portion having a central body comprising a core and at least one pair of diametrically opposite lateral ribs extending radially outwardly from said body past a maximum radial span of said finger of said front applicator portion, said ribs comprise at least one pair of substantially parallel elongated longitudinal fins spanning an axial length of said body of said intermediate applicator portion;
a flared skirt extending radially outwardly from the base of the front applicator portion past the diameter of the diametrically opposite ribs and diverging in a direction away from the tip of the front applicator portion;
a circular rib-engaging disc providing an intermediate circular flange abutting against the rib and positioned between the handle and the rib;
said intermediate applicator portion cooperating with said ribs, skirt, and disk for providing at least two longitudinal chambers defining pockets extending substantially the length of the body of the intermediate applicator portion for receiving material from said container;
said handle being coaxially aligned with said front and intermediate applicator portions and having a length greater than said front intermediate portion, the length of said handle being greater than an axial length of said intermediate applicator portion, said handle being generally flat with a thickness less than a maximum diameter of said intermediate applicator portion and less than a maximum lateral span of said front applicator portion, said handle comprising a rounded end section, a flared section diverging towards said intermediate applicator section, and a manually graspable central section disposed between and connecting said rounded end section and said flared section, said central section having a maximum lateral span less than said rounded end section and said flared section of said handle; and
a circular handle-engaging disc having a diameter greater than a maximum width of said handle, said circular handle-engaging disc having a handle-engaging surface for abuttingly engaging said flared section of said handle and having an intermediate applicator portion-facing surface providing a seat for engaging and supporting said cap.

17: An applicator assembly in accordance with claim 16 including an intermediate base flange providing a grommet disposed between said handle-engaging disc and said rib-engaging disc, and said grommet having a diameter greater than said rib-engaging disc.

18: An applicator assembly in accordance with claim 16 wherein:
said applicator device and said cap comprise different materials;
said applicator device comprises a material selected form the group consisting of a resilient material, an elastomeric material, flexible material, plastic, and rubber;
said cap comprises plastic or metal;
said composition further comprises a topical analgesic with therapeutic benefits for use in humans, equine, bovine, canine, feline, porcine, and other animals and birds for treatment and healing of ligaments, tendons, muscles, bones, hematomas, nerves, and sports injuries and for epidermal and dermal treatment of arthritis, neuralgia and pain;
said pharmaceutical product including a pharmaceutical preparation with antiviral and/or antimicrobial properties for herpes related microbial infections selected from the group consisting of cold sores, herpes simplex virus, varicella zoster virus (herpes zoster) (shingles), and cytomegalovirus, and/or for use in the treatment of a viral disease selected from the group consisting of herpetic keratitis, conjunctivitis, viral influenza (flu), herpes simplex virus 1 (HSV 1), herpes simplex virus 2 (HSV 2), esptein barr, papilloma virus, viral parainfluenza, adenovirus, viral encephalitis, viral menigitus, arbovirus, arenavirus, picornavirus, coronavirus, syntialvirus, viral infections, cormon colds caused by viruses, human immunedeficiency virus (HIV), HIV retinopathy, geniculate zoster, chicken pox, roseola infantum, organ transplant rejection, pneumonia, infectious mononucleosis, uveitis, retinitis, human cervical carcinoma, vaginal carcinoma, vulvovaginitis, human herpes IV, and Kaposi's sarcoma; or said pharmaceutical product comprises a muscle relaxant; and
said medicine comprising a medicine for inhibiting HIV, herpes, viral infections or other infectious diseases in human beings or said medicine comprising a veterinary medicine for treating viral or bacterial infections or infectious diseases in dogs, cats, birds, horses, cows, sheep, swine (pigs or hogs), or other farm animals, rodents, or animals in zoos.
